(19) United States
(12) Patent Application Publication
(10) Pub. No.: US 2025/0197659 A1
(43) Pub. Date: Jun. 19, 2025

(54) A BIOINK FOR BIOPRINTING A HYDROGEL STRUCTURE, SAID HYDROGEL STRUCTURE AND RELATED METHODS

(71) Applicant: Agency for Science, Technology and Research, Singapore (SG)

(72) Inventors: Weijie Cyrus BEH, Nanos (SG); Wan Ling WONG, Proteos (SG); Theresa SEAH, Proteos (SG)

(21) Appl. No.: 18/844,638

(22) PCT Filed: Mar. 7, 2023
(86) PCT No.: PCT/SG2023/050135

§ 371 Date: Sep. 6, 2024

(30) Foreign Application Priority Data
Mar. 8, 2022 (SG) 10202202313X

(51) Int. Cl. C09D 11/04; C09D 11/101; C12N 5/00

(52) U.S. Cl. CPC C09D 11/04; C09D 11/101; C12N 5/0068

(57) ABSTRACT

There is provided a bioink for bioprinting a porous three-dimensional hydrogel structure, the bioink comprising an aqueous medium; and granular crosslinkable hydrogel precursor particles suspended in the aqueous medium, wherein the granular crosslinkable hydrogel precursor particles have an average size of from 100 microns to 500 microns, and wherein under suitable crosslinking conditions, the granular crosslinkable hydrogel precursor particles crosslink and adhere to one another, to form the porous three-dimensional hydrogel structure having pore diameters in the range of from 20 microns to 200 microns. There is also provided a method of forming a porous three-dimensional hydrogel structure using the bioink disclosed herein and a porous three-dimensional hydrogel structure obtained from said method.

### TECHNICAL FIELD

The present disclosure relates broadly to a bioink for bioprinting a hydrogel structure. The present disclosure also relates to the hydrogel structure and a method of making the hydrogel structure.

### BACKGROUND

One of the fundamental challenges in bioprinting is the need to provide cells within the bioprinted structure with an adequate supply of nutrients. In the absence of an active circulation system, bioprinted structures depend on diffusion processes to sustain the cells. While this is sufficient for cells close to a media-facing surface, cell viability becomes adversely affected after just a few hundred microns from the surface. This is because the diffusion of nutrients is limiting, and beyond that distance, the rate of diffusion is unable to keep up with the metabolic requirements of the cells. As a result, even when known bioprinting processes are not ostensibly harmful to cells, the cells within the printed structure starve and die after a few days.

Although introducing channels into printed structures can help to sustain cells deep in a centimeter-scale structure, the cell viability still decreases away from the channel lumen, thus limiting the density of cells that can survive and thrive even in the presence of channels.

In view of the above, there is a need to address or at least ameliorate the above-mentioned problems. In particular, there is a need to provide a bioink for bioprinting a hydrogel structure, a bioprinted hydrogel structure and a method of bioprinting the hydrogel structure that can enhance cell viability throughout the bioprinted structure.

### SUMMARY

According to one aspect, there is provided a bioink for bioprinting a porous three-dimensional hydrogel structure, the bioink comprising an aqueous medium; and granular crosslinkable hydrogel precursor particles suspended in the aqueous medium, wherein the granular crosslinkable hydrogel precursor particles have an average size of from 100 microns to 500 microns, and wherein under suitable crosslinking conditions, the granular crosslinkable hydrogel precursor particles crosslink and adhere to one another, to form the porous three-dimensional hydrogel structure having pore diameters in the range of from 20 microns to 200 microns.

In one embodiment, the granular crosslinkable hydrogel precursor particles comprise one or more of gelatin, alginate, or derivatives thereof.

In one embodiment, the granular crosslinkable hydrogel precursor particles comprise gelatin methacrylate.

In one embodiment, the bioink further comprises an initiator to facilitate crosslinking of the granular crosslinkable hydrogel precursor particles.

In one embodiment, the initiator comprises a photoinitiator, optionally wherein the photoinitiator is lithium phenyl-2,4,6-trimethylbenzoylphosphinate.

In one embodiment, the aqueous medium comprises a cation, optionally wherein the cation is Ca²⁺.

In one embodiment, the granular hydrogel precursor particles further comprise cells, microorganisms or combinations thereof encapsulated therein.

According to another aspect, there is provided a method of forming a porous three-dimensional hydrogel structure, the method comprising dispensing into a volume space, a bioink comprising an aqueous medium and granular crosslinkable hydrogel precursor particles suspended in the aqueous medium, wherein the granular crosslinkable hydrogel precursor particles have an average size of from 100 microns to 500 microns; and crosslinking and allowing the granular crosslinkable hydrogel precursor particles to adhere to one another, thereby forming a porous three-dimensional hydrogel structure having pore diameters in the range of from 20 microns to 200 microns.

In one embodiment, prior to the step of crosslinking the granular crosslinkable hydrogel precursor particles, the method further comprises extruding a sacrificial material into the bioink to form hydrogel fibers within the bioink.

In one embodiment, after the step of crosslinking the granular crosslinkable hydrogel precursor particles, the method further comprises removing the hydrogel fibers to create channels in the hydrogel structure.

In one embodiment, the step of crosslinking the granular crosslinkable hydrogel precursor particles comprises applying ultraviolet light, and optionally heat at a temperature of no more than 32° C.

In one embodiment, removing the hydrogel fibers comprises removing cations from the bioink.

In one embodiment, removing cations from the bioink comprises adding a cation chelator to the bioink.

In one embodiment, the granular crosslinkable hydrogel precursor particles comprise one or more of gelatin, alginate, or derivatives thereof.

In one embodiment, the granular crosslinkable hydrogel precursor particles comprise gelatin methacrylate.

In one embodiment, the bioink further comprises an initiator to facilitate crosslinking of the granular crosslinkable hydrogel precursor particles.

In one embodiment, the initiator comprises a photoinitiator, optionally wherein the photoinitiator is lithium phenyl-2,4,6-trimethylbenzoylphosphinate.

In one embodiment, the cations comprise Ca²⁺.

According to another aspect, there is provided a porous three-dimensional hydrogel structure obtained from the method of disclosed herein, the hydrogel structure comprising, granular hydrogel precursor particles having an average size of from 100 microns to 500 microns that are crosslinked and adhered to one another, wherein spaces between the crosslinked granular hydrogel precursor particles result in pores in the hydrogel structure with pore diameters in the range of from 20 microns to 200 microns.

In one embodiment, the hydrogel structure further comprises one or more channels with a length of from 300 microns to 800 microns and wherein the channels assume the shape of hydrogel fibers that have been removed from the hydrogel structure.

### Definitions

The term “bioink” as used herein is to be interpreted broadly to refer to any biomaterial (e.g. natural or synthetic polymer) that has favourable rheological properties suitable for use in bioprinting. The bioink is typically biocompatible and may contain characteristics that support living cells, facilitate their adhesion, facilitate their proliferation and/or facilitate their differentiation.

The term “bioprinting” as used herein is to be interpreted broadly to refer to an additive manufacturing/deposition process to print an object (e.g. a three-dimensional object) using biomaterials. The printed object is typically used in conjunction with biological systems which include living entities such as cells, tissues etc, or in medical applications. Bioprinting may also involve directly printing with biomaterials that incorporate living entities such as cells, tissues etc.

The term “biocompatible” as used herein is to be interpreted broadly to refer to the ability of a material to perform its intended function without inducing significant inflammatory response, immunogenicity, or cytotoxicity to native cells, tissues, or organs.

The term “cell,” as used herein, is to be interpreted broadly to include individual cells, cell lines, primary cultures, or cultures derived from such cells unless specifically indicated. The cells may be human cells, animal cells, mammalian cells or cells of microorganisms such as yeast, fungi, bacteria etc but is not limited as such.

The term “hydrogel” as used herein is to be interpreted broadly to refer to a network of hydrophilic polymers that are cross-linked via covalent or non-covalent bonds. Due to the hydrophilic nature of hydrogel constituents, hydrogels swell by absorbing water in an aqueous solution but do not dissolve because of a crosslinking structure thereof.

The term “substrate” as used herein is to be interpreted broadly to refer to any supporting structure.

The term “micro” as used herein is to be interpreted broadly to include dimensions from about 1 micron to about 1000 microns, about 1 micron to less than about 1000 microns, about 1 micron to about 900 microns, about 1 micron to about 800 microns, about 1 micron to about 700 microns, about 1 micron to about 600 microns, about 1 micron to about 500 microns, about 1 micron to about 400 microns, about 1 micron to about 300 microns, about 1 micron to about 200 microns, or from about 1 micron to about 100 microns.

The term “nano” as used herein is to be interpreted broadly to include dimensions less than about 1000 nm, about 1 nm to less than about 1000 nm, about 1 nm to about 900 nm, about 1 nm to about 800 nm, about 1 nm to about 700 nm, about 1 nm to about 600 nm, about 1 nm to about 500 nm, about 1 nm to about 400 nm, about 1 nm to about 300 nm, about 1 nm to about 200 nm, or from about 1 nm to about 100 nm.

The term “particle” as used herein broadly refers to a discrete entity or a discrete body. The particle described herein can include an organic, an inorganic, a composite particle or a biological particle. The particle used described herein may also be a macro-particle that is formed by an aggregate of a plurality of sub-particles or a fragment of a small object. The particle of the present disclosure may be spherical, substantially spherical, or non-spherical, such as irregularly shaped particles or ellipsoidally shaped particles. The term “size” when used to refer to the particle broadly refers to the largest dimension of the particle. For example, when the particle is substantially spherical, the term “size” can refer to the diameter of the particle; or when the particle is substantially non-spherical, the term “size” can refer to the largest length of the particle.

The terms “coupled” or “connected” as used in this description are intended to cover both directly connected or connected through one or more intermediate means, unless otherwise stated.

The term “associated with”, used herein when referring to two elements refers to a broad relationship between the two elements. The relationship includes, but is not limited to a physical, a chemical or a biological relationship. For example, when element A is associated with element B, elements A and B may be directly or indirectly attached to each other or element A may contain element B or vice versa.

The term “adjacent” used herein when referring to two elements refers to one element being in close proximity to another element and may be but is not limited to the elements contacting each other or may further include the elements being separated by one or more further elements disposed therebetween.

The term “and/or”, e.g., “X and/or Y” is understood to mean either “X and Y” or “X or Y” and should be taken to provide explicit support for both meanings or for either meaning.

Further, in the description herein, the word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. The intention of the above specific disclosure is applicable to any depth/breadth of a range.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

### DESCRIPTION OF EMBODIMENTS

Exemplary, non-limiting embodiments of a bioink for bioprinting a hydrogel structure, the hydrogel structure and a method of making the hydrogel structure are disclosed hereinafter.

### BIOINK

There is provided bioink for bioprinting a porous three-dimensional hydrogel structure. In various embodiments, the bioink comprises an aqueous medium; and granular crosslinkable hydrogel precursor particles suspended in the aqueous medium. Under suitable crosslinking conditions, the granular crosslinkable hydrogel precursor particles may crosslink and adhere to one another to form the porous three-dimensional hydrogel structure. Suitable crosslinking conditions include but is not limiting to providing a stimulus (e.g. UV irradiation, and/or heat etc) and/or a crosslinking agent/crosslinking promoter/crosslinking initiator (e.g. a covalent crosslinking agent such as APS/TEMED (initiator), glutaraldehyde, etc) to facilitate the crosslinking.

In various embodiments, therefore, the granular crosslinkable hydrogel precursor particles are used as building blocks to construct the porous three-dimensional hydrogel structure. Thus, in these embodiments, the granular crosslinkable hydrogel precursor particles will remain as part of the porous three-dimensional hydrogel structure once crosslinked. This is opposed to the case where hydrogel precursors are merely used to from hydrogel supporting structures or scaffolds to assist the formation another three-dimensional structure, where after the three-dimensional structures are completed, these hydrogel supporting structures or scaffolds are later removed (i.e. do not form part of the final three-dimensional structures).

In various embodiments, the porous three-dimensional hydrogel structure is a highly porous structure. This is because the granular crosslinkable hydrogel precursor particles will naturally have gaps between them. The pores of the porous three-dimensional hydrogel structure can form interconnected “channels” or “network of flow paths” throughout the hydrogel structure. As a result of these gaps or pores, media can easily flow through these gaps or pores, resulting in much better nutrient access for the cells that may be in or on the hydrogel precursor particles or three-dimensional hydrogel structure. Advantageously, the high porosity of the structure allows it to support cell growth and cell viability throughout the structure by facilitating easy access to the nutrients. In addition, the porosity of the structures also permits secondary metabolites to be released quickly into the surrounding (e.g. culture broth).

In various embodiments, the porous three-dimensional hydrogel structure has a porosity ranging from about 10% to about 50%, about 15%, about 20%, about 25%, about 30%, about 35%, about 40%, about 45% or about 50%. As will be appreciated, the porosity of a porous structure may be quantified by measuring the volume fraction that is occupied by the pores. Thus, as an example, in a 1cmx1cmx1 cm hydrogel block with a porosity of about 10% to about 50%, between about 500 to about 900 microliters of the block is occupied by the gel particles, with spaces between these particles occupying the remaining volume.

In various embodiments, the porous three-dimensional hydrogel structure contains pores with pore diameters in the range of from about 20 microns to about 200 microns, in the range of from about 25 microns to about 190 microns, in the range of from about 30 microns to about 180 microns, in the range of from about 35 microns to about 170 microns, in the range of from about 40 microns to about 160 microns, in the range of from about 45 microns to about 150 microns, in the range of from about 48 microns to about 140 microns, in the range of from about 42 microns to about 130 microns, in the range of from about 44 microns to about 120 microns, in the range of from about 46 microns to about 110 microns, or in the range of from about 50 microns to about 100 microns. Advantageously, embodiments of the disclosed porous structure are far superior (in terms of supplying nutrient supply to cells in the hydrogel) to hydrogel structures devoid of such pores. This is because the spaces between the granule particles are large enough such that culture media can flow into them easily. As a result, nutrients travel into the porous hydrogel structure by convection, instead of diffusion. Convection is a much faster process which occurs almost instantaneously, as opposed to hours on a gel without such pores. At the scale of the microparticles, the media can then diffuse quickly into the gel particles, which, at about 100-500 microns, is within the limit of diffusion, typically considered to be around 500 microns. It will be appreciated that in various embodiments, the size of the pores depends on the size and shape of the particles. In general, as a non-limiting illustration, for 300 microns particles that are prepared, the spaces between them may range from about 50-200 microns. However, it should be noted that the size of the pores can in fact be scaled or adjusted (approximately proportionately) according to the particle size.

In various embodiments, the granular crosslinkable hydrogel precursor particles have an average size of from about 100 microns to about 500 microns, from about 150 microns to about 450 microns, from about 200 microns to about 400 microns or from about 250 microns to about 350 microns. It will be appreciated that the size of particles can be customised by using suitable manufacturing techniques or equipment. For example, in various embodiments where the granular crosslinkable hydrogel precursor particles are made by using filter mesh with pores, the size of particles can be customized by using filter mesh with pores of varying diameter. Thus, in this example, the particle size range can be increased by changing the size of the mesh. It should also be noted that although very large particles for encapsulation purposes are not typically used (due to diffusion limits), large particles may nevertheless be useful for preparing porous scaffolds where cells are seeded on the particles (not in the particles).

In various embodiments, hydrogel precursor particles may be obtained by dicing, cutting or blending from a larger hydrogel precursor block. For example, a larger hydrogel precursor block may be cut into smaller particles or blended before being forced through small holes/apertures on a template (e.g. a metal or wire mesh such as a stainless steel mesh with about 300 micron diameter holes) to obtain the hydrogel precursor particles of the desired size. In various embodiments, particles may also be formed by creating droplets or emulsions from melted hydrogel precursor, which are then cooled or otherwise crosslinked into beads. The resulting beads may then be used to as the granular material.

Advantageously, in various embodiments, the configuration and form of granular hydrogel precursor particles make them suitable for use as a bioink and for bioprinting. The granular hydrogel precursor particles may be used in a 3D printer for printing of a 3D structure. Advantageously, in various embodiments, the configuration and form of granular hydrogel precursor particles also make them suitable using handling easy and convenient, for example, they can be pipetted using a standard liquid handler. The ease and convenience of handling the granular hydrogel precursor particles may be useful in certain applications, for example when co-culturing of cells or microbes is desired. In such applications, co-culturing the different granular hydrogel precursor particles containing the different cells or microbes may be performed in a combinatorial fashion more easily and conveniently.

In various embodiments, the granular crosslinkable hydrogel precursor particles are in the form of a gel-like particles. The gel-like particles may be used to create a non-Newtonian suspension bath, into which another material may be deposited. Thus, in various embodiments the form and nature of the granular crosslinkable hydrogel precursor particles allow another material to be easily deposited into their midst, thereby advantageously opening up the possibility of obtaining complex structures, for example, through deposition of a sacrificial material within a volume of granular crosslinkable hydrogel precursor particles before subsequently removing said sacrificial material.

It will be appreciated that hydrogels can be formed using either monomers (e.g., acrylic acid, methacrylic acid), or macromonomers (e.g., gelatin methacrylate, PEG-diacrylate). Non-limiting examples of polymers or co-polymers that are suitable for forming a hydrogel include polyacrylates, polymethacrylates, polyacrylamides, polymethacrylamides, polyvinylpyrrolidone and copolymers thereof. Other examples include polyethers, polyurethanes, and poly(ethylene glycol), functionalized by cross-linking groups or usable in combination with compatible cross linking agents. In various embodiments, the polymer chains may be modified with reactive groups such as acrylates or methacrylates in order for the polymer chains to be photocrosslinkable. In various embodiments, the hydrogel precursors may comprise macromolecules including but not limited to modified polycaprolactone, gelatin, gelatin methacrylate, alginate, alginate methacrylate, modified chitosan, chitosan methacrylate, glycol chitosan, glycol chitosan methacrylate, modified hyaluronic acid (HA), HA methacrylate, and other non-crosslinked natural or synthetic polymeric chains and the like. In various embodiments, the macromolecules may be modified to make the macromolecules crosslinkable. For example, covalent crosslinks may be formed using acrylates, methacrylates, or other types of conjugation chemistry. In various embodiments, the hydrogel precursor disclosed herein may comprise gelatin methacrylate (GelMA) and/or alginate methacrylate (ALMA).

In various embodiments, therefore, the granular crosslinkable hydrogel precursor particles may comprise one or more of gelatin-based particles, alginate-based particles, collagen-based particles, agarose-based particles, collagen-based particles or the like. The granular crosslinkable hydrogel precursor particles may comprise one or more of gelatin or its derivatives (e.g. gelatin methacrylate, gelatin methacryloyl, gelatin methacrylamide etc), alginate or its derivatives (alginate methacrylate, calcium alginate, sodium alginate, etc), hyaluronic acid or its derivatives (e.g. methacrylated hyaluronic acid, thiolated hyaluronic acid), collagen or its derivatives, Methyl cellulose or its derivatives, chitosan or its derivatives, chitin or its derivatives, synthetic peptides and polyethylene glycol or combinations thereof. It will be appreciated that the list of granular crosslinkable hydrogel precursor particles provided above is exemplary and non-limiting. In various embodiments, the materials that may be used for as granular crosslinkable hydrogel precursor particles typically possess at least two crosslinking modalities (i.e. crosslinked in at least two ways/steps); the first modality will allow the particles/granules to be formed, while the second modality will allow the particles to be bound together into the hydrogel block. Non-limiting examples of such dual-crosslinkable materials include acrylated or methacrylated alginate, gelatin, agarose, hyaluronic acid or combinations thereof.

In various embodiments, the bioink may comprise only one single type of granular hydrogel precursor particles or a combination of different types of granular hydrogel precursor particles. When a combination of different types of granular hydrogel precursor particles are used, the combination may offer variability or flexibility in adjusting the physical properties of formed porous three-dimensional hydrogel structure. For example, the porous three-dimensional hydrogel structure may be customised to have the desirable, chemical, biological, optical, structural or physical properties based on the composition of the different granular hydrogel precursor particles. As another example, the different granular hydrogel precursors particles may also have different solubility properties (before or after crosslinking) such that removal of some of these particles in the appropriate solvent/solution (before or after crosslinking) may allow fine-tuning of the pore sizes of the final porous three-dimensional hydrogel structure. In yet another example, control of porosity may be achieved by mixing a first type of hydrogel precursor particles (e.g. non-methacrylated gelatin particles) with a second type of hydrogel precursor particles (e.g. GelMA particles), and melting the first type of hydrogel precursor particles (or its crosslinked form) away after covalent crosslinking of the second type of hydrogel precursor particles.

In various embodiments, the aqueous medium comprises a buffer solution. The buffer solution may include but not limited to a saline solution such as PBS or the like. The aqueous medium may also further comprise additives, ingredients or nutrients that support cell culture, growth, or viability. For example, such additive/ingredients that support cell culture may include but are not limited to serum, amino acids, vitamins, inorganic salts, glucose, and serum as a source of growth factors, hormones, physiological salts, buffers and attachment factors.

Accordingly, the aqueous medium may comprise popular cell culture medium such as MEM, DMEM, RPMI-1640, IMDM, EGM or the like. Advantageously, the presence of ingredients that support cell culture is particularly helpful when the bioink further comprises cells (e.g., encapsulated in the granular crosslinkable hydrogel precursor particles) and/or if there is a desire to later seed cells into the three-dimensional hydrogel structure.

In various embodiments, the bioink further comprises an initiator to facilitate crosslinking of the granular crosslinkable hydrogel precursor particles. For example, the initiator may be a photoinitiator that creates reactive species that facilitates crosslinking of the granular crosslinkable hydrogel precursor particles upon exposure to radiation or light such as ultraviolet light. Non-limiting examples of photoinitiators include benzophenones (aromatic ketones) such as benzophenone and methyl benzophenone; acylphosphine oxide type photo-initiators such as diphenyl (2,4,6 trimethylbenzoyl) phosphine oxide; benzoins and bezoin alkyl ethers such as benzoin, benzoin methyl ether and benzoin isobutyl ether, lithium phenyl-2,4,6-trimethylbenzoylphosphinate (LAP), sodium phenyl-2,4,6-trimethylbenzoylphosphinate (NAP), 2-hydroxy-1-[4-(2-hydroxyethoxy)phenyl]-2-methyl-1-propanone (e.g., Irgacure® 2959), 1-hydroxy-cyclohexyl-phenyl-ketone (e.g., Irgacure® 184), 2,2-dimethoxy-1,2-diphenylethan-1-one (e.g., Irgacure® 651) or the like.

In various embodiments, the initiator may be a thermal initiator that is activated by thermal energy or heat. An example of such thermal initiators include the thermal initiator system comprising ammonium persulfate (APS)-N,N,N′, N′-tetramethylethylenediamine (TEMED) redox pair.

In various embodiments, the concentration of initiator is in the range of about 0.05% to about 10% by weight of the entire bioink. The concentration of initiator may be in the range of, for example, about 0.05% to about 10%, about 0.065% to about 9%, about 0.08% to about 8%, about 0.095% to about 7%, about 0.11% to about 6%, and about 0.125% to about 5%.

Accordingly, a stimulus that results in the crosslinking the granular crosslinkable hydrogel precursor particles may be provided. The stimulus may be light (e.g., UV light) and/or heat. The stimulus may thus be applied by subjecting the bioink to radiation, for example UV radiation and/or infra-red radiation.

The aqueous medium may also comprise a crosslinker that facilitates crosslinking of the hydrogel precursors disclosed herein, including the hydrogel precursor that forms the hydrogel fibers (e.g. for formation of channels later). The crosslinker may include calcium chloride, calcium sulfate, calcium carbonate, calcium (Ca²⁺), magnesium (Mg²⁺), strontium (Sr²⁺), barium (Ba²⁺), and combination thereof. In various embodiments, the crosslinking method for the fibers used in channel formation is reversible. Such methods of crosslinking may be thermal (as in Pluronic F127, gelatin), through ionic crosslinking (e.g. alginate and multivalent cations), or even thiol chemistry.

In various embodiments, the aqueous medium comprises a cation ion. Preferably, the cation is one facilitates crosslinking of another hydrogel precursor (e.g. sacrificial material) that is different from the granular crosslinkable hydrogel precursor particles. In various embodiments, the cation, upon removal may also reverse the crosslinking of the hydrogel precursor (e.g. sacrificial material) that is different from the granular crosslinkable hydrogel precursor particles. In various embodiments, the cation is a metal cation. In various embodiments, the metal cation is Ca²⁺. In various embodiments, the concentration of the cations (e.g. Ca²⁺) is in the range of from about 10 mM to about 500 mM, about 20 mM, about 30 mM, about 40 mM, about 50 mM, about 60 mM, about 70 mM, about 80 mM, about 90 mM, about 100 mM, about 150 mM, about 200 mM, about 250 mM, about 300 mM, about 350 mM, about 400 mM, about 450 mM, or about 500 mM.

In various embodiments, the aqueous medium may contain the crosslinker and/or cation initially, or may not initially contain the crosslinker and/or cation but the cation is later be added prior to dispensing of another hydrogel precursor into the bioink (or porous 3D hydrogel structure or a bath of granular crosslinkable hydrogel precursor particles). That is, the crosslinker and/or cation responsible for crosslinking the other hydrogel precursor (e.g. into hydrogel fibers for forming channels later) may be added into the porous 3D hydrogel structure or a bath of granular crosslinkable hydrogel precursor particles before dispensing the other hydrogel precursor thereinto.

In various embodiments, the bioink further comprises cells, microorganisms, organoids or combinations thereof. For example, the cells, microorganisms (e.g. microbes, bacteria, fungi, yeast or the like) and/or organoids may be seeded onto or distributed among the surface of the granular hydrogel precursor particles or encapsulated therein. The same or different cells, microorganisms and/or organoids may be provided in the bioink. For example, by encapsulating different microbes into separate preparations of the hydrogel precursor particles, it is possible to perform co-culturing experiments by mixing the different hydrogel precursor particles, and crosslinking them to bring different microbes together.

In various embodiments, the bioink may be at an acidic pH, at a neutral pH or at a basic pH. In various embodiments, the bioink may be at a physiological pH of about 7.4. It will be appreciated that the working temperature for the bioink depends on the material used for the hydrogel particles. In general, if co-printing with cells, the temperature is preferably maintained between about 4-37° C. Otherwise, if cells are introduced after crosslinking is complete, the temperature range may generally be limited only to the thermal stability of the materials, and freezing/boiling temp of water. In various embodiments, the bioink may be used for bioprinting at a temperature falling in the range of from about 4° C. to about 37° C., about 5° C., about 10° C., about 15° C., about 20° C., about 25° C., about 30° C., or about 35° C. In various embodiments, the bioink may be sterile. In various embodiments, the bioink may be biocompatible. In various embodiments, the bioink may be non-toxic. In various embodiments, the bioink may have a viscosity suitable for dispensing from a bioprinter and/or a pipette.

### Method of Forming a Porous Three-Dimensional Hydrogel Structure

There is also provided a method of forming a porous three-dimensional hydrogel structure. In various embodiments, the method comprises dispensing into a volume space (e.g. to form a bath), a bioink disclosed herein (e.g. one comprising an aqueous medium and granular crosslinkable hydrogel precursor particles suspended in the aqueous medium); and crosslinking and allowing the granular crosslinkable hydrogel precursor particles to adhere to one another, thereby forming a porous three-dimensional hydrogel structure. In various embodiments of the method, the bioink, crosslinkable hydrogel precursor particles, aqueous medium, porous three-dimensional hydrogel structure, and/or stimulus comprise(s) one or more of the characteristics discussed earlier above.

In various embodiments, prior to the step of crosslinking the granular crosslinkable hydrogel precursor particles (e.g. before applying a stimulus to the bioink for crosslinking), the method further comprises extruding a sacrificial material that is different from the material of the granular crosslinkable hydrogel precursor particles (e.g. a different hydrogel precursor), into the bioink to form hydrogel fibers (e.g. crosslinked hydrogel fibers) within the bioink. These hydrogel fibers may serve as sacrificial constructs that may be removed subsequently. In various embodiments, the sacrificial material is one that has a different mode of crosslinking compared to the granular crosslinkable hydrogel precursor particles. For example, the sacrificial material may crosslink based on a stimulus, factor and/or crosslinker that is different from the stimulus, factor and/or crosslinker that crosslinks the granular crosslinkable hydrogel precursor particles. The sacrificial material may also be non-reactive towards the stimulus, factor and/or crosslinker that crosslinks the granular crosslinkable hydrogel precursor particles. Accordingly, the method may utilize different modes of (e.g., multi-modalities) crosslinking to achieve the final three-dimensional hydrogel structure (e.g. through different materials and/or different crosslinking mechanisms). In various embodiments, the sacrificial material is reversibly crosslinkable, for example, the sacrificial material may cross-link upon exposure to a factor, stimulus and/or crosslinker and reverse its crosslinking after removal of the factor, stimulus and/or crosslinker. In various embodiments, the sacrificial material crosslinks to form a hydrogel fiber upon exposure or contact with a cation ion (e.g. a metal cation such as a Ca²⁺).

As the crosslinked hydrogel fibers may serve as sacrificial constructs/structures, the arrangement of one or more hydrogel fibers can serve as a mold/template for an arrangement of one or more channels in the porous 3D hydrogel structure. The sacrificial fibers may be straight or curved. The arrangement of one or more sacrificial fibers may be arranged in a regular pattern or a non-regular pattern. The one or more sacrificial structures may be shaped and sized according to a predetermined design and may serve as a mold/template for various structures in the porous 3D hydrogel structure. The sacrificial structures on their own may also be three-dimensional and spans across different planes of the porous 3D hydrogel structure. In various embodiments, the hydrogel fibers or sacrificial fibers are in a semi-solid or gel state. Advantageously, the gel state helps the hydrogel fibers or sacrificial fibers to maintain its shape and form in the bath of bioink such that the sacrificial material does not mix with the bioink and does not substantially lose its shape and form during formation of the porous 3D hydrogel construct.

In various embodiments, the sacrificial material and the granular crosslinkable hydrogel precursor particles may be independently derived from one or more of the hydrogel precursors disclosed herein. For example, they may be independently selected from the group consisting of gelatin-based precursors, alginate-based precursors, collagen-based precursors, agarose-based precursors, collagen-based, hyaluronic acid-based precursors or the like. In various embodiments, the sacrificial material is an alginate-based precursor while the granular crosslinkable hydrogel precursor particles are gelatin-based precursor particles. In various embodiments, the granular crosslinkable hydrogel precursor particles are made with acrylated or methacrylated gelatin, agarose, and/or alginate while the sacrificial material is made with Pluronic F127, agarose, alginate, and/or gelatin. In various embodiments, the sacrificial material is alginate while the granular crosslinkable hydrogel precursor particles are crosslinkable gelatin-methacrylate particles. Alginate molecules are capable of reversibly crosslinking in the presence of multivalent cations such as calcium while gelatin-methacrylate molecules are capable crosslinking to in the presence of UV light/radiation. Accordingly, in various embodiments, the GelMA/Alginate combination is used to create an ink system that can be crosslinked in two steps-first physically and reversibly (using calcium/alginate), then covalently and irreversibly (using UV to crosslink the GelMA). In alternative embodiments, the sacrificial material may also be a thermally-reversible material or hydrogel such as Pluronic F127, gelatin or the like. Such thermally reversible material may be reversibly solidified at a particular temperature and reversibly liquefied at another temperature. Other types of reversible hydrogels may also be used as the sacrificial material.

In various embodiments, after the step of crosslinking the granular crosslinkable hydrogel precursor particles (e.g. after applying the stimulus to the bioink which crosslinks the granular crosslinkable hydrogel precursor particles), the method further comprises removing the hydrogel fibers (e.g. formed from sacrificial material and serve as a sacrificial fiber or construct) to create channels in the porous three-dimensional hydrogel structure. In various embodiments, the final porous three-dimensional structure contains defined channels but is devoid of the sacrificial material (i.e. different material from the granular hydrogel precursor particles) and the hydrogel fibers formed therefrom. Advantageously, the presence of the channels may further enhance accessibility of nutrients within the porous three-dimensional hydrogel structure. Even more advantageously, the channels provide a way to interface with host circulation should the porous three-dimensional hydrogel structure be implanted, while the porosity can serve as a way to template the microvascularization process. In various embodiments, the channels provide defined flow paths through the already-porous hydrogel block. This can serve the purposes of creating interfaces (such as inlets and outlets). As the positions of these channels can be specified in the design of the 3D model, this provides the ability to couple the flow to external ‘fluidics’, including host vasculature when the porous three-dimensional hydrogel structure is implanted.

The channels created by the removed crosslinked hydrogel fibers may have lengths in the range of from about 300 microns to about 800 microns, from about 350 microns to about 750 microns, from about 400 microns to about 700 microns, from about 450 microns to about 650 microns, from about 400 microns to about 600 microns or about 500 microns.

The step of removing the hydrogel fibers (or sacrificial fibers/structure/construct) to create channels in the hydrogel structure may comprise removing the stimulus and/or factor and/or crosslinker that results in the formation of crosslinked hydrogel fibers (formed from the sacrificial material). In various embodiments, the step of removing the hydrogel fibers (or sacrificial fibers/structure/construct) may comprise liquefying the sacrificial hydrogel fibers to facilitate its removal from the porous 3D hydrogel structure. In various embodiments, the step of liquefying the hydrogel fibers (or sacrificial fibers/structure/construct) depends on the type of sacrificial material used. For example, the sacrificial material may be liquefied using a physical approach (for example, by changing the temperature of the sacrificial material), a chemical approach (for example, by modifying/changing the chemical composition of the sacrificial material), or a combination thereof. In various embodiments, removing the crosslinked hydrogel fibers comprises removing cations (e.g. Ca²⁺) from the bioink and/or the porous three-dimensional hydrogel structure. The removal of cations from the bioink may comprise using a cation chelator or sequester (e.g. Ca²⁺ chelator such as citrate, EDTA, EGTA or the like).

In various embodiments, the step of crosslinking the granular crosslinkable hydrogel precursor particles comprises applying a stimulus such as ultraviolet light and/or heat to the bioink. In various embodiments, the method further comprises apply heat to the granular crosslinkable hydrogel precursor particles before and/or during crosslinking, for example, at a temperature of no more than about 32° C., at a temperature of from about 25° C. to about 32° C., at a temperature of from about 26° C. to about 32° C., at a temperature of from about 27° C. to about 32° C., at a temperature of from about 28° C. to about 32° C., at a temperature of from about 29° C. to about 32° C. or at a temperature of from about 30° C. to about 32° C. Advantageously, in various embodiments, raising the temperature slightly (e.g. below 32° C. such as about 27° C.) before exposure to UV to cause the granular crosslinkable hydrogel precursor particles to melt slightly (but not completely) which would promote adhesion of the granular crosslinkable hydrogel precursor particles to one another.

In various embodiments, there is also provided a method of preparing a porous hydrogel structure having channels, the method comprising providing a granular medium, wherein the granular medium comprises a first hydrogel precursor in a cation-containing buffer and wherein the first hydrogel precursor comprises granular crosslinkable particles; extruding a sacrificial material (e.g. a second hydrogel precursor) into the granular medium to form/print hydrogel fibers within the granular medium; applying a stimulus to the granular medium to stabilize/obtain a porous hydrogel structure; and removing the hydrogel fibers to create channels in the porous hydrogel structure.

### Porous Three-Dimensional Hydrogel Structure

There is also provided a porous three-dimensional hydrogel structure disclosed herein. In various embodiments, the porous three-dimensional hydrogel structure is obtained from the method disclosed herein.

In various embodiments, the porous three-dimensional hydrogel structure comprises granular hydrogel precursor particles that are crosslinked and adhered to one another. The granular hydrogel precursor particles may have one or more characteristics discussed earlier above. For example, the granular hydrogel precursor particles may have an average size of from about 100 microns to about 500 microns. In various embodiments, the spaces between the crosslinked granular hydrogel precursor particles result in pores in the hydrogel structure with pore diameters in the range of from about 20 microns to about 200 microns. In various embodiments, the hydrogel structure further comprises one or more channels with a length of from about 300 microns to about 800 microns and wherein the channels assume the shape of hydrogel fibers that have been removed from the hydrogel structure.

In various embodiments, porous three-dimensional hydrogel structure further comprises cells, microorganisms (e.g., microbes, bacteria, fungi, yeast or the like) and/or organoids disposed thereon or therein.

In various embodiments, the porous three-dimensional structure may contain more than one type of hydrogels (i.e. formed from two or more different types of hydrogel precursors). In various embodiments, when the porous three-dimensional structure contains more than one type of hydrogels (i.e. formed from two or more different types of hydrogel precursors), the structural characteristics or form of each of the different hydrogel may be the similar or different (e.g. one may have a discontinuous structure made up of granular particles while the other different hydrogel may be of a substantially homogenous and continuous phase.).

In various embodiments, the porous three-dimensional structure contains only a single type of hydrogel (formed from a single type of hydrogel precursor) but is devoid of a second different type of hydrogel (formed from a sacrificial material or a second different type of hydrogel precursor).

### BRIEF DESCRIPTION OF FIGURES

FIG. 1A is a schematic showing crosslinkable GelMA particles (102) in a calcium bath (104) in accordance with an embodiment (100) disclosed herein.

FIG. 1B is a schematic in accordance with the embodiment (100) of FIG. 1A showing that the crosslinkable GelMA particles (102) support the extrusion by an extrusion nozzle (106) of another material such as alginate (108) in their midst, which quickly crosslinks into hydrogel fibers (110).

FIG. 1C is a schematic in accordance with the embodiment (100) of FIG. 1B showing that the exposure to UV light (112) causes the GelMA particles (102) to crosslink covalently, making them stable even at elevated temperatures.

FIG. 1D is a schematic in accordance with the embodiment (100) of FIG. 1C showing after exposure to UV radiation, the individual particles (102) also adhere to each other. The resulting hydrogel then contains channels (114) through which media (116) can be introduced, which, when coupled with the highly porous nature of the hydrogel, can supply ample nutrient to the cells.

FIG. 2A and FIG. 2B are photographs (200) of a channel formed by alginate fibers labelled with blue dye (204) in the GelMA granular medium (202) in accordance with an embodiment disclosed herein. The alginate fibers served as sacrificial ink and was removed subsequently leaving the blue dye (204) in the formed channel. However, the porosity of the medium makes the channel difficult to visualize, since the blue food dye (204) within the channel would quickly permeate the entire structure. Instead, in FIG. 2B, EDTA stained with red food dye (206) is carefully pipetted on top (208) of the channels, and the dye (206) can be seen to flow into the channel as it clears out.

FIG. 3A-FIG. 3C are a series of photographs showing the permeability of the porous hydrogel formed in accordance with an embodiment disclosed herein.

FIG. 3A shows a block (205) of porous hydrogel structure formed based on embodiments of methods disclosed herein. FIG. 3B shows the block (206) of porous hydrogel structure of FIG. 3A when blue coloured dye (208) is pipetted (210) on top of the block (206). The block (206) of porous hydrogel structure formed with granular media is filled rapidly with blue food dye (208). FIG. 3C shows the block (206) of porous hydrogel structure of FIG. 3B completed permeated by the blue dye (208), highlighting the high permeability of the porous hydrogel structure obtained. In similar fashion, when the porous hydrogel structure is used as a scaffold for cells, nutrients from the cell culture media can reach the cells quickly.

FIG. 4 are cross-sectional views of human umbilical vascular endothelial cells (HUVECs) seeded on porous hydrogel block in accordance with an embodiment disclosed herein. In this embodiment, a channel is formed in the porous hydrogel block by removing the F127 sacrificial fiber, after photocrosslinking of the granular hydrogel precursor particles to form the porous hydrogel block. HUVECs were then seeded on the block, and seen to completely cover the particle surfaces. Section thickness is around 200 microns. Scale bar=200 microns.

### EXAMPLES

Example embodiments of the disclosure will be better understood and readily apparent to one of ordinary skill in the art from the following discussions and if applicable, in conjunction with the figures. It should be appreciated that other modifications related to structural, chemical and biological changes may be made without deviating from the scope of the invention. Example embodiments are not necessarily mutually exclusive as some may be combined with one or more embodiments to form new exemplary embodiments.

In the following examples, it is shown that sub-millimeter-sized, reactive granular gel particles (e.g. gelatin methacrylate) in a suitable aqueous medium or buffer (henceforth referred to as granular medium) can be covalently crosslinked together to create highly porous structures, which permit easy access to nutrients and are thus conducive to cell growth. Using a photoinitiator, the granular medium can be photocrosslinked, and even patterned using 3D printing. Cells can be seeded onto the surface of the porous gel, or be encapsulated within the gelatin particles. Organoids can also be distributed among the granular medium, and be embedded in the resulting structure. The granular medium also permits channels to be formed using sacrificial materials.

### Example 1-Granular Medium for Creating Porous Hydrogel Structures

In this example, granular particles out of crosslinkable gelatin methacrylate (GelMA), and a photoinitiator (LAP) are created. The granular bioink is prepared by first dissolving GelMA (from Gelomics) in culture medium or buffer to a final concentration of 20% wt/v, followed by the addition of 2 mM photoinitiator Lithium phenyl-2,4,6-trimethylbenzoylphosphinate (LAP). The gelMA is cooled at 4° C. for 30 mins for physical gelation to occur, then cut into small pieces and resuspended in buffer containing 2 mM LAP. The suspended gelMA is loaded into a syringe and syringed through a stainless-steel mesh (Cytiva Lifesciences), resulting in a gelMA slurry with particles with an average diameter of 300 um.

It will be appreciated that it is possible to manipulate the size of the granular particles for their specific applications by customizing the diameter of the pores in the steel mesh. The size of the pores determines what can be loaded onto the gel after crosslinking. Conversely, it also determines what can be trapped within the gel structures. This is particularly useful for organoid printing. When organoids (100-600 microns in size) are mixed into the granular bioink, and crosslinked into the structure, they will not fall out of the structure during culture because they are larger than the gaps. On the other hand, cells (5-10 microns) are small enough to be seeded after crosslinking is completed. This can be a useful way to introduce vessel-forming cells into the structure to induce vascularization of the hydrogels.

The GelMA used has a gel strength of 300 g bloom, with degree of substitution of from about 40% to about 80%. The bloom strength and the degree of substitution may be adjusted to tweak the physical properties of the resulting hydrogel as desired, which in turn affect cellular differentiation and proliferation.

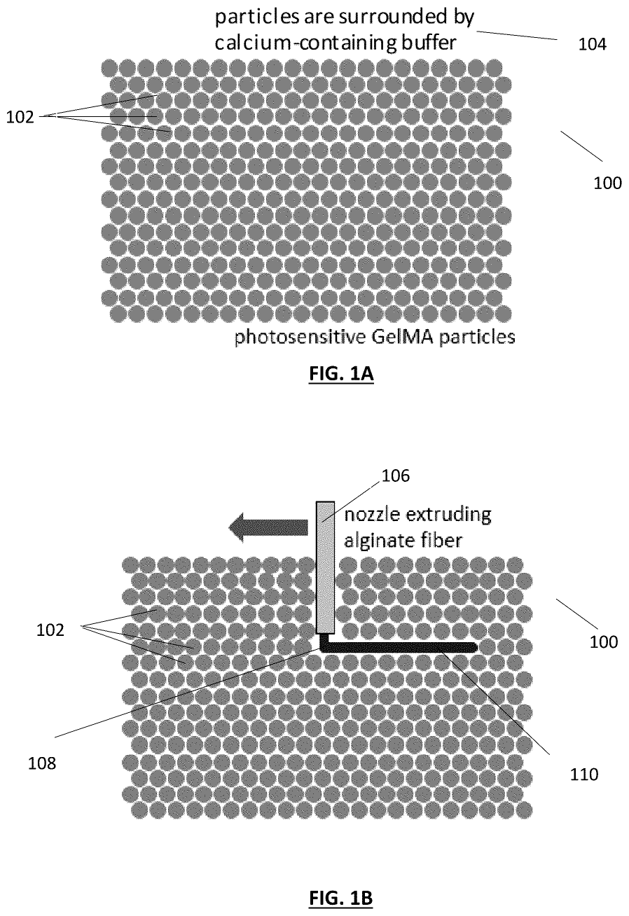

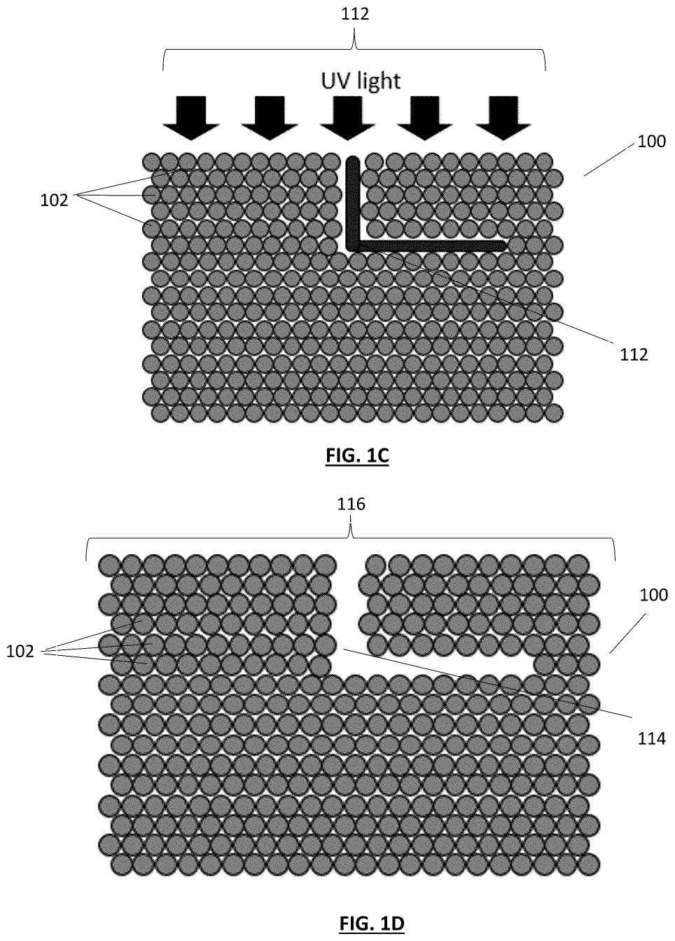

In most respects, the GelMA particles behave identically to gelatin. By suspending the GelMA particles in an aqueous medium containing 50 mM calcium, a different hydrogel precursor (such as alginate) can be optionally extruded into the bath be crosslinked into hydrogel fibers (see FIG. 1A and FIG. 1B).

It will be appreciated that the ingredients in the aqueous medium/buffer may be varied depending on the cells to be cultured as well as the type of sacrificial material used. For example, for alginate sacrificial fibers, 50 mM calcium may be added to the aqueous medium/buffer, which may be PBS, cell culture medium, or any other aqueous buffer, supplemented with LAP (e.g. 2 mM).

The suspension of granular GelMA particles in aqueous medium are then loaded into a 3D bioprinter for printing of a 3D structure. The bioprinting of the 3D structure may be carried out manually or via a computer implemented method for e.g. based on a predetermined design file loaded in a bioprinting software.

After the 3D structure is printed using the granular GelMA particles, the granular medium is exposed to UV light. The presence of the photoinitiator results in crosslinking of the GelMA medium. Not only do the individual particles crosslink covalently, they also adhere to each other, resulting in interconnected particle clusters. This adhesion can also be further promoted by raising the temperature slightly (e.g. 27° C.) before exposure to UV, which would cause the GelMA to melt slightly. Care is taken to avoid excessive heating, however, as raising it beyond around 32° C. will result in complete melting of the GelMA. After UV crosslinking, the hydrogel particles of the 3D structures no longer melt, even at elevated temperatures.

### Example 2-Forming Channels within the Porous Hydrogel Structures

Channels may also be formed within the porous hydrogel structures if desired. These channels provide a way to interface with host circulation should the 3D porous hydrogel structure be later implanted in a subject.

Briefly, to form channels within the porous hydrogel structures, blended granular gelatin-based particles in the form of GelMA (containing a photoinitiator) are deposited/extruded into a volume space (e.g. printing of a 3D hydrogel structure) so as to create a non-Newtonian suspension bath, into which another material (such as alginate) can be deposited. This other material will later serve as a sacrificial material. The bath is supplemented with calcium ions, which allows the alginate to crosslink. As the gelatin-based particles do not flow easily, the newly-formed alginate hydrogel fiber does not sink, but is instead spatially-suspended in the bath of granular GelMA particles (or 3D hydrogel structure).

More specifically, in this Example, 2 mL granular gelMA bioink (formed using the same methods described in Example 1) is pipetted into a 2 cm×1 cm×1 cm mold at room temperature and exposed to a light source of wavelength of 405 nm (Thor Labs M405LP1-C1) for 5 mins. Channels are then introduced into the porous hydrogel structure. Channels are created in the porous block, by adding 100 mM calcium chloride (Sigma), and extruding 2% alginate (medium viscosity, Sigma) fibers into the granular slurry, using a 23 G needle.

Like in Example 1, the granular GelMA is then exposed to UV light. The presence of the photoinitiator results in crosslinking of the GelMA particles.

After photocrosslinking, the GelMA particles become locked in place (see FIG. 1C). At this point, the sacrificial alginate fibers can be cleared by immersion into chelators such as citrate or EDTA (Gibco), whereupon the calcium will be sequestered, and the alginate hydrogel will dissolve (See FIG. 1D).

The resulting structure contains a combination of channels (˜ 500 microns) and highly porous matrix (pores ˜50-100 microns). The channels provide a way to interface with host circulation should the structure be implanted, while the porosity can serve as a way to template the microvascularization process.

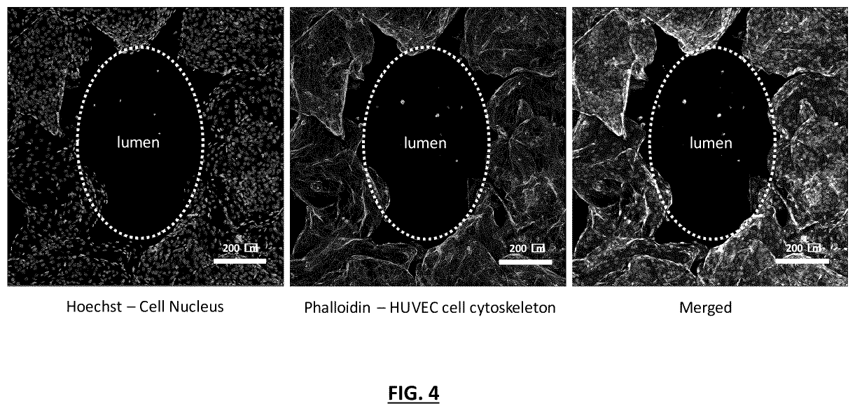

As an alternative to alginate/calcium fibers, other reversible crosslinking material may be used to create sacrificial channels. For example, thermally-reversible hydrogel fibers such as Pluronic F127 or gelatin (not GelMA) can be used to form the channels. Pluronic F127 fibers can be deposited at 25-37° C., and removed by lowering the temperature to about 4° C. For example, the lumen in FIG. 4 was formed using F127 fibers. Alternatively, warm gelatin (not GelMA) solution can also be deposited into cool or room temperature granular medium bath, and melted away by raising the temperature after covalent crosslinking of the granular medium. More specifically in these cases, the granular bioink bath may be maintained at a suitable temperature to ensure that the extruded material will form fibers (e.g. above 20° C. for F127, below 28° C. for gelatin). After photocrosslinking, the fibers are liquefied by changing the temperature (e.g. chilled to 4° C. for F127, and warmed to 32° C. for gelatin fibers) to form the channels. It should be appreciated that the above materials illustrated for creating sacrificial channels are not exhaustive and other types of reversible hydrogels can also be used.

The inventors note that the granular particles can contract during the crosslinking process, which affects how the channel-forming fibers (which don't contract) are arranged in the structure. Thus, the crosslinking and hydrogel preparation process may be further controlled to minimize the change in size.

### Example 3-Demonstrating High Porosity of the Structure

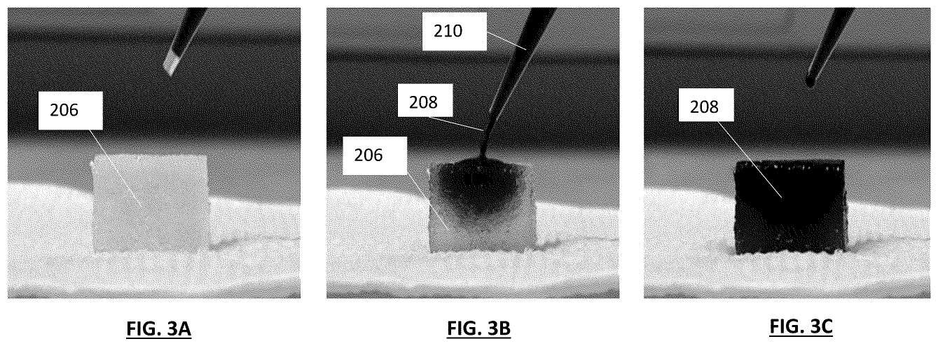

To demonstrate the porosity of the granular medium (or formed granular 3D hydrogel structure), an experiment is conducted where a rectangular structure is fabricated using the GelMA granular medium. After crosslinking, the structure is transferred onto a napkin to wick away the suspension bath. The resulting structure is a porous hydrogel, into which at least 400 microliters (or around 20% of the volume of the entire structure) of blue food dye can be rapidly loaded as shown in FIG. 3A-3C. Since the dye flows into the spaces between the particles, instead of diffusing in, the rate of mass transfer is very high. Accordingly, cells seeded onto such a porous structure can remain fully accessible to the surrounding media, and nutrients can be easily replenished, thus maintaining good cell viability.

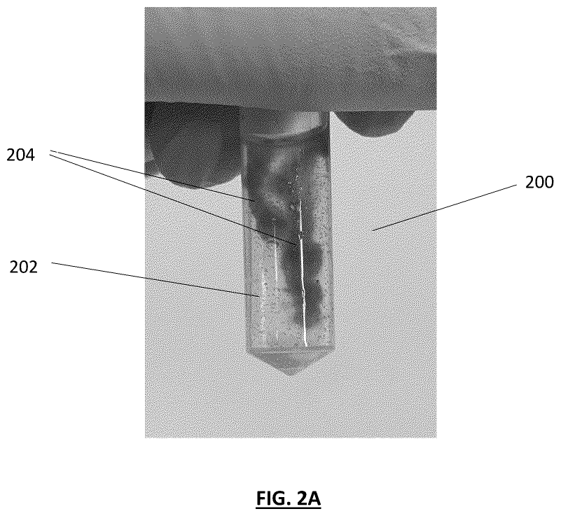

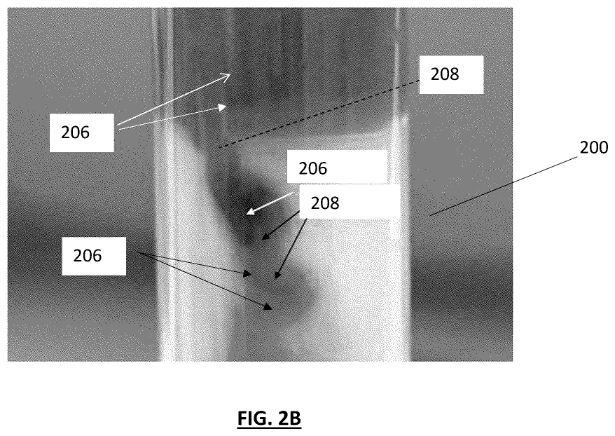

The ability to form channels inside the granular medium was described previously in Example 2. To demonstrate the porosity of the granular medium in a porous 3D hydrogel structure containing channels, another experiment is conducted by depositing into the medium, alginate fibers which are dyed blue when forming channels in the medium. Due to the porosity of the medium, the blue dye rapidly disperses, causing the subsequently formed channels to look larger than they really are (as shown is FIG. 2). When EDTA with red dye is carefully pipetted on top of the crosslinked medium, the red dye can be seen to move into the channel (as shown is FIG. 2).

To demonstrate the ability to support high density culture of cells on these hydrogel blocks formed from granular hydrogel precursor particles disclosed in embodiments herein, a further experiment is conducted. In this experiment, human umbilical vascular endothelial cells are seeded into hydrogel blocks, formed from granular hydrogel precursor particles disclosed herein. In this experiment, a channel was additionally formed in the hyper-porous hydrogel block using F127 sacrificial fibers. After seeding, the cells were cultured for 24 hours to allow cell attachment to take place. The samples were then fixed, stained, sectioned, and imaged (see FIG. 4). It is worth noting that the high porosity allows easy seeding of a large numbers of cells on these granular particles, since the cell-containing medium can flow through the gaps between the particles, and the cells can be distributed throughout the hydrogel block. Cells have been cultured for more than a week under such conditions, with no evidence of nutrient inadequacy.

Thus, the above experiments show that the 3D hydrogel structures formed with or without channels therein are highly porous and allow nutrients to be easily and effectively accessed by cells seeded therein.

In conclusion, the examples above utilize photosensitive microparticles as the bioink to produce a porous hydrogel structure. As these particles will naturally have gaps between them, media can easily flow through these gaps, resulting in much better nutrient access for the cells that may be in or on these microparticles. For example, a simple immersion of the porous hydrogel structure in culture media is sufficient to supply the cells with ample nutrients. Furthermore, the ability to form channels using extruded, sacrificial fibers such as alginate, which allow us to create the additional feature of defined flow paths through the already-porous hydrogel block. This serves the purposes of creating interfaces (such as inlets and outlets). As the positions of these channels can be specified in the design of the 3D model, this allows coupling of the flow to external ‘fluidics’, including host vasculature when implanted.

It should also be highlighted that in bioprinting, it is usually the intention to achieve high resolution and as such, the nozzle sizes tend to be very small. Accordingly, granular materials are generally hard to extrude using standard bioprinters and therefore is typically not a material of choice for bioprinting. Using granular materials for bioprinting is hence completely against conventional wisdom. However, contrary to conventional wisdom, the inventors have surprisingly found out that bioprinting with granular materials is in fact not detrimental, but yet can significantly address or ameliorate the problems of insufficient perfusion of nutrients in existing bioprinted structures as the granular materials allows three-dimensional structures to be printed with an unexpectedly high level of porosity. Moreover, using granular materials as a printing medium requires the adaptation of the bioprinter's nozzle size to accommodate the extrusion of granular materials, and this advantageously opens up the possibility of allowing organoids to be directly deposited as well during printing. It was also surprisingly found that there were no significant disadvantages in the obtained bioprinter structure despite the loss of fine print resolution through the use of larger bore nozzles.

### Applications

Various embodiments disclosed herein provide a reactive, granular hydrogel material that comprises sub-millimeter-sized gel particles in an appropriate buffer (i.e. granular medium) which: is able to support the deposition of soft materials to create complex structures within it; can be covalently crosslinked to create highly porous gel structures; can be patterned by 3D printing using e.g. photocrosslinking; can encapsulate cells in individual gel particle; and/or can be pipetted, thus permitting combinations of cells (e.g. fungi, bacteria) to be co-cultured.

In various embodiments, the highly porous hydrogel created using the disclosed granular medium provides a unique way to encapsulate or seed cells in the 3D structure (e.g. a centimeter-sized block), while maintaining easy access to nutrients in the culture media throughout the entire structure. This has beneficial applications in bioprinting. For example, cells can either be encapsulated in the particles (e.g. GelMA), seeded onto a porous structure, or be introduced in the form of organoids. The cells are able to remain viable while the structure matures, e.g. when vascularization takes place, either through the introduction of endothelial cells, or using growth factors like VEGF, or both.

In various embodiments, the highly porous structure of the hydrogel blocks formed using granular material disclosed herein also permits high-density culture of adherent cells, which is of interest to virus production (e.g. using HEK293T to produce lentiviral vectors), antibody production using mammalian cell factories, and the cultured meat industry, where fat and muscle cells need to be added to their products. The ability to perform this culture on an immobile substrate like the porous three-dimensional hydrogel structure disclosed herein is advantageous over suspension cultures, which require agitation in large bioreactors, and which cannot achieve the same cell density as embodiments of the hydrogel structure disclosed herein.

In addition to biomedical applications that use mammalian cells, embodiments of the granular medium disclosed herein may also have use in fermentation processes in natural product biosynthesis. In particular, co-culturing of microbes (e.g. fungi and bacteria) is known to induce production of different metabolites. By encapsulating microbes in the granular particles (e.g. GelMA particles), mixing different microbe-laden granular medium together, and crosslinking them, the resulting porous gel blocks/structure is expected to be able to serve as useful co-culturing platforms. In this case, the porosity not only permits nutrients to reach the cells, but also allow the metabolites secreted by the cells to enter the culture media. Furthermore, as various embodiments of the granular medium disclosed herein can be pipetted using large-bore tips, the medium may be more easily and conveniently manipulated using automated liquid handlers. By combining different microbes, it is possible to create different co-culturing porous gel blocks that can be placed in a single pot, thereby simplifying and streamlining the co-culturing process.

It will be appreciated by a person skilled in the art that other variations and/or modifications may be made to the embodiments disclosed herein without departing from the spirit or scope of the disclosure as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

1. A bioink for bioprinting a porous three-dimensional hydrogel structure, the bioink comprising:
an aqueous medium; and
granular crosslinkable hydrogel precursor particles suspended in the aqueous medium,
wherein the granular crosslinkable hydrogel precursor particles have an average size of from 100 microns to 500 microns, and
wherein under suitable crosslinking conditions, the granular crosslinkable hydrogel precursor particles crosslink and adhere to one another, to form the porous three-dimensional hydrogel structure having pore diameters in the range of from 20 microns to 200 microns.

2. The bioink of claim 1, wherein the granular crosslinkable hydrogel precursor particles comprise one or more of gelatin, alginate, or derivatives thereof.

3. The bioink of claim 2, wherein the granular crosslinkable hydrogel precursor particles comprise gelatin methacrylate.

4. The bioink of claim 1, wherein the bioink further comprises an initiator to facilitate crosslinking of the granular crosslinkable hydrogel precursor particles.

5. The bioink of claim 4, wherein the initiator comprises a photoinitiator, optionally wherein the photoinitiator is lithium phenyl-2,4,6-trimethylbenzoylphosphinate.

6. The bioink of claim 1, wherein the aqueous medium comprises a cation, optionally wherein the cation is Ca²⁺.

7. The bioink of claim 1, wherein the granular hydrogel precursor particles further comprise cells, microorganisms or combinations thereof encapsulated therein.

8. A method of forming a porous three-dimensional hydrogel structure, the method comprising:
dispensing into a volume space, a bioink comprising an aqueous medium and granular crosslinkable hydrogel precursor particles suspended in the aqueous medium, wherein the granular crosslinkable hydrogel precursor particles have an average size of from 100 microns to 500 microns; and
crosslinking and allowing the granular crosslinkable hydrogel precursor particles to adhere to one another, thereby forming a porous three-dimensional hydrogel structure having pore diameters in the range of from 20 microns to 200 microns.

9. The method of claim 8, wherein prior to the step of crosslinking the granular crosslinkable hydrogel precursor particles, the method further comprises extruding a sacrificial material into the bioink to form hydrogel fibers within the bioink.

10. The method of claim 9, wherein after the step of crosslinking the granular crosslinkable hydrogel precursor particles, the method further comprises removing the hydrogel fibers to create channels in the hydrogel structure.

11. The method of claim 8, wherein the step of crosslinking the granular crosslinkable hydrogel precursor particles comprises applying ultraviolet light, and optionally heat at a temperature of no more than 32° C.

12. The method of claim 10, wherein removing the hydrogel fibers comprises removing cations from the bioink.

13. The method of claim 12, wherein removing cations from the bioink comprises adding a cation chelator to the bioink.

14. The method of claim 8, wherein the granular crosslinkable hydrogel precursor particles comprise one or more of gelatin, alginate, or derivatives thereof.

15. The method of claim 14, wherein the granular crosslinkable hydrogel precursor particles comprise gelatin methacrylate.

16. The method of claim 8, wherein the bioink further comprises an initiator to facilitate crosslinking of the granular crosslinkable hydrogel precursor particles.

17. The method of claim 16, wherein the initiator comprises a photoinitiator, optionally wherein the photoinitiator is lithium phenyl-2,4,6-trimethylbenzoylphosphinate.

18. The method of claim 12, wherein the cations comprise Ca²⁺.

19. A porous three-dimensional hydrogel structure obtained from the method of claim 8, the hydrogel structure comprising,
granular hydrogel precursor particles having an average size of from 100 microns to 500 microns that are crosslinked and adhered to one another,
wherein spaces between the crosslinked granular hydrogel precursor particles result in pores in the hydrogel structure with pore diameters in the range of from 20 microns to 200 microns.

20. The porous three-dimensional hydrogel structure of claim 19, wherein the hydrogel structure further comprises one or more channels with a length of from 300 microns to 800 microns and wherein the channels assume the shape of hydrogel fibers that have been removed from the hydrogel structure.
